(19) United States
(12) Patent Application Publication
(10) Pub. No.: US 2007/0244045 A1
(43) Pub. Date: Oct. 18, 2007

(54) Treatment of Acute Respiratory Distress Syndrome

(76) Inventors: Bruce Holm, Rochester, NY (US); Edmund A. Egan, Buffalo, NY (US)

Correspondence Address: JAECKLE FLEISCHMANN & MUGEL, LLP, 190 Linden Oaks, ROCHESTER, NY, 14625-2812, US

(21) Appl. No.: 11/626,128

(22) Filed: Jan. 23, 2007

Related U.S. Application Data
(60) Provisional application No. 60/761,250, filed on Jan. 23, 2006.

(51) Int. Cl. A61K 38/17; A61P 11/00

(52) U.S. Cl. 514/12

(57) ABSTRACT

Patients suffering from acute respiratory distress syndrome or acute lung injury are treated by administering to the patients a therapeutically effective dosage of a surfactant that includes SP-B and phospholipid at a concentration of SP-B relative to concentration of phospholipid that is sufficient to produce detectable SP-B dependent activity.

### CROSS REFERENCE TO RELATED APPLICATION

This application claims priority to U.S. Provisional Patent Application No. 60/761,250, filed Jan. 23, 2006, and to PCT/US2006/02423, filed Jan. 23, 2006.

### FIELD OF INVENTION

The present invention relates to the treatment of a patient suffering from acute lung injury (ALI) and/or acute respiratory distress syndrome (ARDS) by administering a lung surfactant preparation to the patient.

### BACKGROUND OF THE INVENTION

Acute respiratory distress syndrome (ARDS) was originally termed the adult respiratory distress syndrome because it resembled the clinical picture of infant respiratory distress syndrome (IRDS), and both exhibited hyaline membranes at autopsy (Lancet. 1967; 2:319-323, Chest. 1971; 60:233-239). Avery and Mead (Am. J. Dis. Child. 1959:97:517-523) first reported that lung surfactant quantity and activity were abnormal in infants with IRDS, and surfactant replacement has subsequently become standard therapy for premature infants at risk for or having IRDS. Petty and Ashbaugh (Chest. 1971; 60:233-239) described qualitative and quantitative surfactant deficiencies in their initial description of ARDS, and the subsequent scientific literature, recently reviewed by Notter (Lung Surfactants. New York, N.Y.: Marcel Dekker, 2000), has supported the role of surfactant dysfunction in both ARDS and less severe acute lung injury (ALI) (Am. J. Respir. Crit. Care Med. 1994; 149:818-824).

Surfactant replacement in ARDS and ALI in humans has been largely unsuccessful. Three large prospective, randomized controlled clinical trials of surfactant replacement demonstrated little or no benefit in adults with ARDS or ALI who were treated with aerosolized synthetic Exosurf (Burroughs Wellcome, Kirkland, Quebec) N. Engl. J. Med. 1996; 334: 1471-1421), instilled semisynthetic Survanta (Abbott Laboratories, Abbott Park, Ill.) (Am. J. Respir. Crit. Care Med. 1997; 144:1309-1315), and instilled recombinant surfactant-specific protein C-based Venticute (ALTANA Pharma, Konstanz, Germany) (N. Engl. J. Med. 2004; 341:884-892).

Surfactant preparations differ in phospholipids, neutral lipid, and protein composition and the failure of previous trials may relate to these differences. The importance of the hydropobic surfactant apoprotein surfactant-specific protein B has only recently been recognized (Pediatr. Res. 1986; 20:460-467).

Calfactant is a modified natural surfactant with a ratio of phospholipids to apoprotein SP-B similar to that found in natural bovine surfactant (Expert Opin. Pharmacother. 2001; 2:149-1493). Biophysical and biological testing demonstrates activity equal to natural surfactant (Am. Rev Respir. Dis. 1992; 145:24-30) and resistance to inhibition by plasma and other proteins associated with lung injury or by cell wall and lysophospholipids (Am. J. Respir. Crit Care Med. 1998; 158:28-35).

It was hypothesized that a natural surfactant containing high levels of SP-B, such as calfactant, might prove effective in ARDS or ALI. A positive acute response to calfactant administration in an open-label trial in 29 children ventilated for ALI was reported in 1996 (Crit Care Med. 1996; 24:1316-1322), and a subsequent controlled but un-blinded study of 42 patients replicated this acute improvement and demonstrated a shortened ventilator and intensive care unit course (Crit. Care Med. 1999; 27:188-195). The positive results in those preliminary studies led to a multicenter, blinded, controlled trial of calfactant compared with placebo in infants, children, and adolescents with respiratory failure from ARDS or ALI.

### SUMMARY OF THE INVENTION

The present invention is directed to a process for treating a patient suffering from lung disease that requires the use of mechanical ventilation to sustain breathing. The process comprises the step of administering to the patient a therapeutically effective dosage of a surfactant comprising SP-B and phospholipid at a concentration of SP-B relative to concentration of phospholipid that is sufficient to produce detectable SP-B activity.

The preferred surfactant is calfactant.

### BRIEF DESCRIPTION OF THE DRAWINGS

FIG. 1 is a chart depicting the enrollment of patients randomly assigned to the calfactant and placebo groups.

FIG. 2 is a graph comparing the successful extubation of calfactant and placebo patients during the study.

FIG. 3 includes two graphs of oxygenation index vs time for the calfactant and placebo patient groups.

FIG. 4 contains Tables 1, 2, and 3, which are discussed in the Detailed Description of the Invention.

### DETAILED DESCRIPTION OF THE INVENTION

The present invention relates to a process for treating a patient suffering from acute respiratory distress syndrome (ARDS) or acute lung injury (ALI), as well as other patients suffering from lung disease that requires the use of mechanical ventilation to sustain breathing but does not meet either the X-ray and/or the severity criteria for ARDS or ALI. More specifically, the invention is directed to the treatment of a patient selected from the group consisting of (a) patients suffering from acute lung injury, who are patients with acute respiratory failure requiring mechanical ventilation, with severe bilateral lung edema/collapse by chest X-Ray, and having a ratio of arterial oxygen partial pressure, PaO₂, to fraction of inspired oxygen, FiO₂, that is less than 300 (PaO₂/FiO₂<300); (b) patients suffering from acute respiratory distress syndrome (ARDS) who are a subset of ALI patients in that their PaO₂/FiO₂<200; and (c) patients suffering from lung disease that requires the use of mechanical ventilation to sustain breathing but does not meet either the X-ray and/or the severity criteria for ARDS or ALI.

The process comprises the step of administering to the patient a therapeutically effective dosage of a surfactant comprising SP-B and phospholipid at a concentration of SP-B relative to concentration of phospholipid that is sufficient to produce detectable SP-B dependent activity, whereby the patient has an improved likelihood of survival relative to a comparable patient treated with a placebo.

With respect to this invention, the term “SP-B” is understood to refer either to “apoprotein SP-B” or “a protein that exhibits Sp-B-like activity.”

In a preferred embodiment of the invention, the surfactant is administered by intratracheal instillation.

In another preferred embodiment of the invention, the therapeutically effective dosage comprises about 10 mg phospholipid/kg body weight to about 200 mg phospholipid/kg body weight, which is equivalent to about 400 mg to about 8400 mg phospholipid/m² of body surface area, or about 11 ml to about 240 ml/m² of a suspesion containing phospholipid in a concentration of about 35 mg/ml.

In another preferred embodiment of the invention, the surfactant comprises a saline suspension comprising about 25 mg/ml to about 100 mg/ml of phospholipid, plus SP-B in an amount of about 0.1 wt. % to about 4.0 wt. %, based on the weight of phospholipid.

In another preferred embodiment of the invention, the surfactant is a lung surfactant.

In a particularly preferred embodiment of the invention, the surfactant is calfactant.

The activity of SP-B can be measured as biophysical activity or biologic activity. Biophysical activity is determined by observing that the surface tension of an inverted air “bubble” in the suspension under consideration reaches <3 mN/m at minimum bubble volume within 5 minutes when oscillated in a Pulsating Bubble Surfactometer (Electronetics, Amherst, N.Y.) at 20 cycles/minute as described in Wang et al. (Am J Physiol Lung Cell Mol Physiol 2002; 283: L897). Biologic activity is determined by observing restoration to normal of the deflation pressure-volume curve in an excised or in situ surfactant deficient animal lung using the method of Bermel (Lung 1984; 162:99-113) or Mizuno (Pediatr Res 1995, 37:271-276).

In another embodiment, the present invention is drawn to the supplementation of the compositions of the invention with exogenous SP-B or SP-C, alone or in combination, and particularly to the supplementation with exogenous SP-B. For example, the results of the present invention are counterintuitive in that ARDS/ALI is not readily characterized as a surfactant deficient state, a term which would be most appropriately used to describe the situation that prevails in the lungs of a premature infant. Instead, while not wishing to be bound by any particular theory, the results obtained herein suggest that it might be most appropriate to refer to ARDS/ALI as a surfactant dysfunctional state, where normal surfactant function is disrupted by, for example, proteins and other materials resulting from lung damage.

Again while not wishing to be bound by any particular theory, the results obtained herein are strongly suggestive of the protective aspects of surfactants in such situations of surfactant dysfunction, and also strongly suggest that the presence of SP-B and/or SP-C, but particularly SP-B, in added surfactant may confer an advantageous effect on a patient suffering from ARDS/ALI, particularly in terms of reduction of patient mortality for such ARDS/ALI patients.

Thus in light of the above, an embodiment of the present invention is based on the supplementation of the surfactants of the invention, including, but not limited to the calf-lung based surfactants of the invention, with exogenous SP-B or SP-C. For example, the surfactants of the invention may be supplemented with SP-B or SP-C obtained as described in U.S. Pat. Nos. 6,020,307 or 6,458,759, herein incorporated in their entireties by reference. Such compositions may exhibit further advantageous properties as described above or in addition to the above. Assays for these properties will include clinical studies as well as physical and biochemical assays for lung function such as described elsewhere herein. Assays will also include animal models which model the presence of various compounds in the lungs in ARDS/ALI cases that may result in surfactant dysfunction, such that any protective effects surfactant containing exogenously provided SP-B or SP-C alone or in combination may be assayed.

In the above embodiment of the invention, SP-B may be added in an amount such that the total amount of SP-B in the surfactant (i.e., endogenous plus exogenous) is about 0.1 wt. % to about 4.0 wt. %, based on the weight of phospholipids, and more preferably in an amount such that the total amount of SP-B in the surfactant is about 0.7 wt %, 0.8 wt %, 0.9 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %. 3.0 wt %, 3.1 wt %, 3.2 wt %, 3.3 wt %, 3.4 wt %, 3.5 wt %, 3.6 wt %, 3.7 wt %, 3.8 wt %, 3.9 wt %, or 4.0 wt %, and most preferably greater than about 0.7 wt % based on the weight of phospholipids. SP-C may be added in similar amounts.

The following description is illustrative of the process of the present invention. Those skilled in the art will recognize that scope of the invention is not limited by the specific examples and treatment protocols described herein.

Patients

In accordance with the present invention, a patient undergoing treatment is preferably post-neonatal, i.e., after 40 weeks post-conceptual age, and one week or more after birth.

Twenty-one pediatric intensive care units (PICUs) across the Pediatric Acute Lung Injury and Sepsis Investigator network enrolled patients over a 3-year period from July 2000 to July 2003. Institutional review boards at each institution approved the study protocol. Informed consent was obtained from a parent or guardian prior to enrollment. Demographic information obtained included age, sex, and race/ethnicity (white, black, Hispanic, or other), determined from the medical record.

Entry criteria included age 1 week to 21 years; respiratory failure due to radiographically evident bilateral parenchymal lung disease; enrollment within 24 hours of initiation of mechanical ventilation (extended to 48 hours after the initial 50 patients); and an oxygenation index higher than 7 [oxygenation index=(fraction of inspired oxygen)×(mean airway pressure)×100/PaO₂].

Exclusion criteria included prematurity (corrected gestational age <37 weeks); status asthmaticus; head injury with Glasgow Coma Scale of <8; chronic lung disease defined by home oxygen or diuretic use; brain death, do not resuscitate orders, ongoing cardiopulmonary resuscitation, or limitation of life support; significant airway disease that might delay extubation; uncorrected congenital heart disease, preexisting myocardial dysfunction, or cardio genic pulmonary edema.

Randomization was stratified to balance the severity of lung injury between groups at study entry. Stratification was based on evidence of increased mortality in patients with an oxygenation index of 13 or higher (fast entry) compared with an oxygenation index higher than 7 but less than 13 (slow entry) within 6 hours of the initiation of mechanical ventilation.

Study Protocol

Patients were randomized to receive intratracheal instillation of 2 doses of 80 mL/m² calfactant (35 mg/mL of phospholipid suspension in saline) or equal volume of air placebo. For infants weighing less than 10 kg, the equivalent newborn dose of calfactant was 3 mL/kg. Treatment was administered in 4 equal aliquots instilled intratracheally via a small catheter. Patient positions were changes between aliquots (left decubitus, head up then down; right decubitus, head up the down) and sedation and neuromuscular blockade were given for the procedure. Gas exchange was maintained by manual ventilation with 100% oxygen using pressures comparable with those previously used on mechanical ventilation. By protocol, a second intervention was performed a mean (SD) of 12 (2) hours later if the oxygenation index remained higher than 7.

To maintain blinding, a pharmacist drew the next (opaque) envelope from the appropriate fast entry or slow entry file previously randomized centrally in blocks of 2 and 4 and sent the syringes of calfactant or placebo to the PICU in an opaque container. A respiratory therapist not otherwise involved with the care of the patient placed opaque tape on the endotracheal tube and performed the intervention. Physicians, investigators, and nurses caring for the patient remained blinded to treatment assignment throughout the study.

Participating physicians agreed to follow ventilator guidelines limiting tidal volume of less than 8 mL/kg; fraction of inspired oxygen of less than 0.6; peak inspiratory pressure of less than 40 mm Hg; and Pa_{CO2} of greater than 40 and less than 60 mm Hg. Blood gases and ventilator settings were evaluated through study day 14.

Treatment with other surfactants was prohibited and the clinical care team determined all other aspects of the patient's care. All data were collected prospectively.

Study Drugs

Calfactant (Infasurf produced by ONY Inc, Amherst, N.Y.) is a modified natural lung surfactant approved by the Food and Drug Administration for IRDS and produced by extracting the phospholipids, neutral lipids, and hydrophobic apoproteins SP-B and SP-C from bovine lung surfactant obtained by saline lavage of newborn calf lungs.

Study Outcome

The primary efficacy outcome was the duration of respiratory failure as measured by ventilator-free days in the 28 days following study entry. A ventilator-free day is a composite outcome that incorporates both mortality and duration of mechanical ventilation. In the analysis, death or the need for exctrcorporeal membrane oxygenation are equivalent to unresolved respiratory failure at 28 days and equal to no ventilator-free days. Death was prospectively identified as the most important outcome and was carefully monitored for safety reasons. Based on mortality differences in preliminary studies, the study was not expected to identify a mortality benefit (Crit Care Med. 1996; 24:1316-1322).

Additional efficacy outcome measurements included PICU and hospital lengths of stay, hospital charges, duration of supplemental oxygen therapy, and failure of conventional mechanical ventilation (defined a priori by the use of high-frequency oscillatory ventilation, nitric oxide, or extracorporeal membrane oxygenation).

The acute effects of surfactant therapy were evaluated by comparing the oxygenation index in the treatment and placebo groups over the 24 hours after treatment. Vital signs and oximetry were monitors continuously and recorded at 5-minute intervals for 30 minutes after the intervention. Complications at the time of study intervention included any significant change in vital signs (e.g., bradycardia, hypotension) or sustained (>30 seconds) oxygen saturation of less than 80%. Safety outcomes included mortality, pulmonary complications (air leaks, pulmonary hemorrhage, and nosocomial pneumonia), and any unexpected adverse events.

Management of the Study

The original study design called for enrollment of 300 patients and completion in 2 years. Sample size calculation based on pilot study data (Crit. Care Med. 1999; 27:188-195) suggested a 25% reduction in the 13-day average ventilator course for pediatric respiratory failure would require 274 patients with an α level of 0.05 and a β level of 0.10. After the first year, it became apparent that participating centers were enrolling fewer patients than expected. The data and safety monitoring board endorsed a 1-year study extension and closure of the study at the end of that year regardless of enrollment. The data and safety monitoring board conducted an interim safety analysis when 100 patients had been enrolled. No significant differences in adverse events or deaths were found. However, mortality was higher than in the previous two studies (Crit Care Med. 1996; 24:1316-1322, Crit. Care Med. 1999; 27:188-195), prompting a blinded review of all deaths by the board. The board concluded that the increase in deaths was due to the inclusion of immunocompromised children in the current study. At the direction of the Food and Drug Administration, the board continued to review the findings with each additional 10 deaths. The study was stopped at the predetermined 3-year limit and was not stopped because of mortality differences. The mortality difference we found was not discovered until after the study was closed.

Statistical Analysis

_{χ}² tests were used to compare groups with respect to categorical outcomes. The Wilcoxon rank sum test was used to compare groups with quantitative outcomes. Cure-rate models were used to compare time with successful extubation (Lung 1984; 162:99-1). Repeated measures models were used to compare the oxygenation index within subjects over time. In post hoc analyses, logistic regression models were used to assess treatment effects on mortality, which were adjusted for fast or slow entry stratification factor; study site (sites with ≦10 patients enrolled were treated as one site); age category (<1 year, 1-5 years, 6-13 years, >13 years); and immune status (immunocompromised vs noncompromised). All variables and the subset of variables found to be significant were then tested in multivariate models that included the treatment group. We used statistical software to fit the cure rate models (GAUSS, Aptech Systems, Kent, Wash.) and for other analyses (SAS version 8.2, SAS Institute, Cary, N.C.). Statistical significance was considered to be P<0.05.

Results

A total of 153 patients provided consent, but a parent withdrew consent prior to treatment. Seventy-seven patients were randomized to the calfactant group, and 75 patients were randomized to the placebo group (FIG. 1). All data were included in an intention-to-treat analysis.

At study entry, 91% of patients met ARDS criteria and all patients met ALI criteria (Am. J. Respir. Crit. Care Med. 1994; 149:818-824). There were no significant differences between groups in demographic profile, severity of illness at randomization, or coexisting diagnoses or co-morbidities (TABLE 1). Although not statistically significant, there were five additional bone marrow transplant patients in the placebo group and three additional near-drowning patients in the surfactant arm; both groups had high baseline mortality. Eight protocol violations were identified: six patients (three placebo and three calfactant) had an initial oxygenation index of less than 7 but met all other entry criteria, and two patients (one placebo and one calfactant) received nonprotocol surfactant administration after the study intervention. Adherence to the ventilator guidelines was comparable between groups. Fraction of inspired oxygen and peak pressures were within guidelines more than 90% of the time and Pa_{CO2} was higher than 40 mm Hg more than 80% of the time.

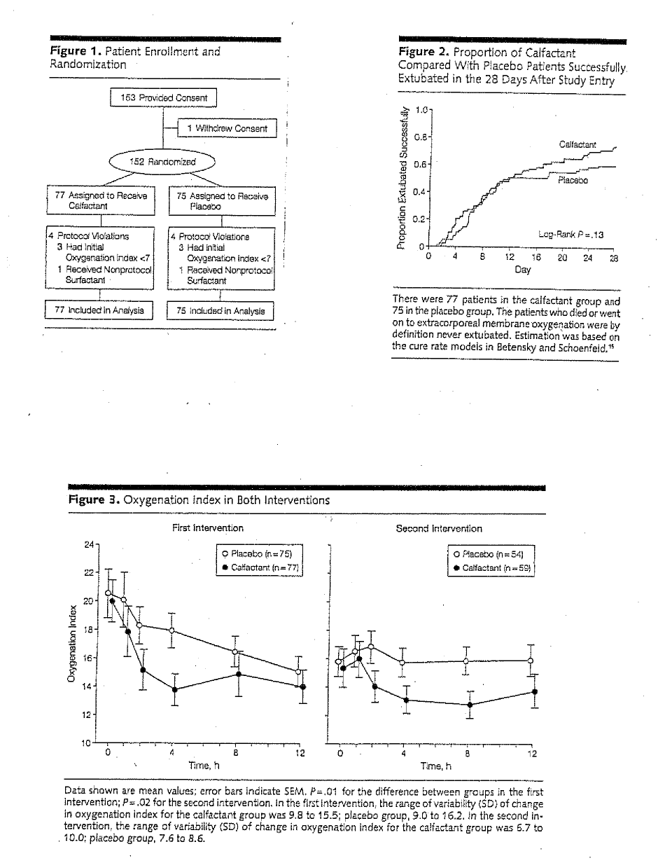

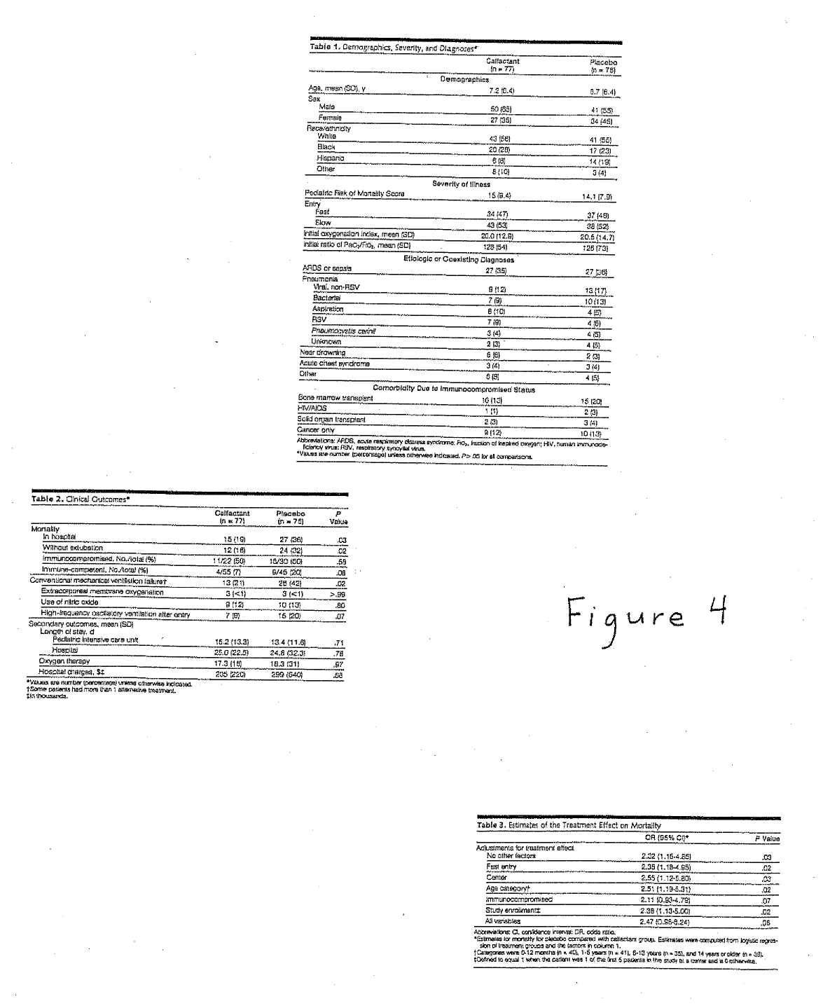

Unexpectedly, mortality was significantly greater in the placebo group compared with the calfactant group (27/75 vs 15/77; odds ratio [OR], 2.32 [95% confidence interval {CI}, 1.15-4.85]) when all deaths were considered and was still significant when death without recovery from respiratory failure was considered (TABLE 2). Respiratory failure was given as the primary cause of death in 40% of patients and as a major contibutor of death in 43% of patients. Calfactant patients averaged a mean (SD) of 13.2 (10) ventilator-free days at 28 days, while placebo patients averaged 11.5 (10.5) ventilator-free days (P=0.21). The cumulative percentages of extubated patients in each group over the first 28 days appear in FIG. 2.

Oxygenation as measured by oxygenation index significantly improved with both doses of calfactant (FIG. 3). Improvement after the first intervention was not adequate to preclude retreatment in most patients, however, as most calfactant (70%) and placebo patients (79%) received a second intervention per the study protocol because their oxygenation index remained greater than 7.

Infants younger than 12 months constituted 26% of the population. Mortality in this subgroup of placebo patients was more than three times that of calfactant-treated patients (9/19 vs 3/21; P=0.02). Ventilator-free days were also statistically fewer in placebo patients (mean [SD], 7.0 [9.9] vs. 15.2 [10.3]; P=0.01)

Table 2 reports other clinical outcomes. More placebo patients did not respond to conventional mechanical ventilation after the study intervention. Comparison of duration of oxygen therapy, hospital and PICU lengths of stay, and hospital charges revealed no statistical differences between groups.

Immediate complications associated with instillation were more frequent in calfactant patients and were similar to the acute responses of new-borns to surfactant instillation (Pediatrics. 1997; 100:31-38). Hypotension was seen in 9% of calfactant instillations compared with 1% of placebo instillations (P=0.005). All patients with hypotension reaponded to volume infusion. Transient hypoxia occurred in 12% of calfactant instillation compared with 3% of placebo instillations (P=0.008), but resolved when the calfactant instillation was slowed and/or the positive-pressure ventilation was transiently increased. No patient was removed from the study because of treatment complications. The incidence of air leaks was 13% in the calfactant group and 16% in the placebo group (P=0.65). Nonsocomial pneumonia was seen in 6% of calfactant patients and 11% of placebo patients (P=0.40). No systemic complications were ascribed to the intervention in either group. The ORs and associated 95% CIs of the treatment effect on mortality adjusted factors identified a priori (fast vs. slow entry, center) or a posteriori (age, immune status, enrollment number) are shown in TABLE 3. Although treatment group is not significant in all models, particularly those that adjust for immunocompromised status, the OR associated with the treatment was at least 2.1 for all models listed in TABLE 3.

Infants, children, and adolescents with ALI who received calfactant in this multicenter study had decreased mortality, more rape improvement in oxygenation index, and were more likely to respond to conventional mechanical ventilation. The primary outcome variable, ventilator-free days, were not significantly different between groups. Transient hypoxia and hypotension were more common with calfactant treatment, but these effects were mild and did not necessitate withdrawal from the study. The acute positive effect of calfactant on ventilation in this trial is consistent with previous studies of calfactant in children (Crit Care Med. 1996; 24:1316-1322, Crit. Care Med. 1999; 27:188-195).

Infant respiratory distress syndrome results from quantitative deficiency of surfactant leading to respiratory failure from progressive atelectasis. Surfactant is also deficient in ARDS and ALI, but is also inhibited by inflammatory mediators, plasma proteins, and cellular debris that are seeping into the airspace. Consequently, the challenges for successful surfactant replacement therapy in ARDS and ALI are more complex than for IRDS (Biometrics. 2001; 57: 282-286). Two surfactants effective in IRDS had disappointing results when tested in large clinical trials in ARDS and ALI (N. Engl. J. Med. 1996; 334: 1417-1421, Am. J. Respir. Crit. Care Med. 1997; 155:1309-1315).

The previously observed acute benefits of calfactant on lung function were replicated herein (Crit. Care Med. 1996; 24:1316-1322, Crit. Care Med. 1999; 27:188-195). Both doses of calfactant improved oxygenation, demonstrating that it can form a functioning film in the injured lung. Calfactant did not, however, restore lung function to normal nor did all of the patients respond positively. Only 55% of calfactant patients (vs. 33% of placebo patients) had a 25% or greater improvement in oxygenation index by 12 hours after the first intervention.

The duration of respiratory failure was not improved with calfactant as it was in a pilot study (Crit. Care Med. 1999; 27:188-195). The average duration of ventilation in calfactant compared with placebo patients was similar (11.3 vs 10.8 days), as were lengths of stay and hospital charges. The absence of benefit in these parameters may be a consequence of the unexpected disproportionate survival of calfactant-treated patients. As was observed with the introduction of surfactant therapy in premature infants, increased survival may actually increase the need for prolonged supportive care (J. Clin. Invest. 1991; 88:1976-1981).

Severity of initial lung injury was expected to influence survival. Mortality rate was indeed higher in fast (37%) compared with slow entry (20%) subgroups. Mortality was lower in both strata for calfactant patients (26% calfactant vs. 46% placebo for fast entry and 14% vs. 26% for slow entry, respectively). Unresolved respiratory failure was given as the primary cause or a major contributor in 83% of deaths, and lack of improvement in oxygenation after the intervention was strongly associated with mortality. Improvement in lung function offers a plausible mechanism whereby calfactant treatment might increase survival because respiratory failure was a significant cause of death in this trial.

Overall mortality in this study was higher than in the pilot study (Crit. Care Med. 1999; 27:188-195) (14% in pilot study vs. 28% herein). attributable to the inclusion of the previously excluded immunocompromised patients whose mortality rate (56%) was four times that of immunocompetent patients (13%). Mortality rates were lower for calfactant patients in both the immunocomprosed (50% vs. 60%) and immunocompetent (7% vs. 20%) subgroups. The numerically greater number of immunocompromised patients in the placebo group (30 in the placebo group vs. 22 in the calfactant group; P=0.17) influenced the observed overall mortality difference between the groups. The ORs for mortality with placebo treatment approached but did not reach statistical significance (P=0.08) after post hoc adjustment for immune status (TABLE 3). This study was not powered sufficiently to detect effects in specific patient subgroups.

There may be multiple reasons for the failure of other surfactants in the three large ARDS trials (N. Engl. J. Med. 1996; 334: 1417-1421, Am. J. Respir. Crit. Care Med. 1997; 155:1309-1315, N. Engl. J. Med. 2004; 351:884-892), but none of these trials used a lung surfactant preparation containing a sufficient amount of SP-B, which is the essential factor for full surfactant activity and resistance of a surfactant to inhibitition by blood and inflammatory protein molecules present in the alveoli in ARDS/ALI (J. Clin. Invest. 1991; 88:1976-1981, Am. J. Respir. Crit. Care Med. 1996; 153:176-184). Congenital absence of SP-B in humans causes lethal neonatal respiratory distress syndrome (J. Clin. Invest. 1994; 91:1860-1863), and mice who are bred deficient of SP-B die at birth of respiratory failure (Proc. Natl. Acad. Sci. USA. 1995; 92: 7794-7798). The SP-B protein by itself confers full biophysical and biological activity on surfactant phospholipids (J Lipid. Res. 1996; 37:1749-1760). The surfactant used in the study has the highest level of resistance to inactivation, as determined by in vitro and in vivo experimental testing, due to its high ratio of SP-B to phospholipids (Expert Opin. Pharmacother. 2001; 2:1479-1493, Am. Rev. Respir. Dis. 1992; 145:24-30, Eur. Respir J. 1993:6:971-977). It has greater surface activity and physiological activity in animal lungs than Exosurf or Survanta, which are two surfactants previously used to treat ARDS in adults (Chem. Phys. Lipids. 2002; 114:21-34). Additionally, the amount of calfactant administered in this trial was more than three times the estimated normal lung surfactant content of 20 mg/kg (Lung Surfactants. New York, N.Y.: Marcel Dekker, 2000).

In this multicenter, randomized, blinded trial, calfactant administration early in the course of pediatric acute respiratory failure results in acute improvement in oxygenation and unexpectedly produced lower mortality. Adverse effects of the therapy were minimal.

What is claimed:

1. A process for treating a patient suffering from lung disease that requires the use of mechanical ventilation to sustain breathing, said process comprising:
the step of administering to said patient a therapeutically effective dosage of a surfactant comprising SP-B and phospholipid at a concentration of SP-B relative to concentration of phospholipid that is sufficient to produce detectable SP-B dependent activity.

2. The process of claim 1 wherein the patient is selected from the group of (a) patients suffering from Acute Respiratory Distress (ARDS), (b) patients suffering from Acute Lung Injury (ALI), and (c) other patients who do not meet the X-Ray and/or severity criteria for ARDS or ALI.

3. The process of claim 1 wherein said surfactant is a lung surfactant.

4. The process of claim 3 wherein said lung surfactant is calfactant.

5. The process of claim 1 wherein said step of administering further comprises the step of administering said surfactant by intratracheal instillation.

6. The process of claim 1 wherein said patient has an age of at least about one week.

7. The process of claim 1 wherein said patient is post-neonatal.

8. The process of claim 1 wherein said therapeutically effective dosage comprises about 10 mg phospholipid/kg body weight to about 200 mg phospholipid/kg body weight.

9. The process of claim 1 wherein the surfactant comprises a saline suspension comprising about 25 mg/ml to about 100 mg/ml of phospholipid, plus SP-B in an amount of about 0.1 wt. % to about 4.0 wt. %, based on the weight of phospholipid.

10. The process of claim 9, wherein the SP-B comprises exogenous SP-b.

11. The process of claim 10, wherein the exogenous SP-B is added such that the total amount of SP-B in the surfactant is at least about 0.7 wt %.

12. A process for treating a patient suffering from lung disease that requires the use of mechanical ventilation to sustain breathing, said process comprising:
the step of administering to said patient a therapeutically effective dosage of a surfactant comprising SP-B and phospholipid at a concentration of SP-B relative to concentration of phospholipid that is sufficient to produce detectable SP-B dependent activity;
wherein said surfactant is calfactant, and wherein said SP-B comprises exogenous SP-B added such that the total amount of SP-B in the surfactant is at least about 0.7 wt %.
